(19) United States
(12) Patent Application Publication
(10) Pub. No.: US 2009/0131329 A1
(43) Pub. Date: May 21, 2009

(54) Treatment for allograft rejection

(76) Inventors: Edmund John Miller, Huntington, NY (US); Shin Hirayama, Toronto (CA); Takeshi Shiraishi, Fukuoka City (JP)

Correspondence Address: AMSTER, ROTHSTEIN & EBENSTEIN LLP, 90 PARK AVENUE, NEW YORK, NY, 10016, US

(21) Appl. No.: 12/283,246

(22) Filed: Sep. 10, 2008

Related U.S. Application Data
(60) Provisional application No. 60/993,768, filed on Sep. 14, 2007.

(51) Int. Cl. A61K 38/08; A61P 37/06; A61K 31/17; A61K 31/7088; A61K 31/7105

(52) U.S. Cl. 514/17; 514/597; 514/44

(57) ABSTRACT

Provided are methods of treating a mammal at risk for allograft rejection. The methods comprise treating the mammal with a compound that reduces an interaction between a CXC chemokine and a CXC receptor.

### CROSS-REFERENCE TO RELATED APPLICATION

This application claims the benefit of U.S. Provisional Patent Application No. 60/993,768, filed Sep. 14, 2007, the content of which is hereby incorporated by reference in its entirety.

### BACKGROUND OF THE INVENTION

(1) Field of the Invention

The present invention generally relates to methods of inhibiting allograft rejection. More specifically, the invention is directed to methods of preventing allograft rejection by inhibiting interactions between CXC chemokines and a CXC receptors.

(2) Description of the Related Art

In the early 1980s lung transplantation was rarely used, but in the last decade it has become a widely available therapeutic option. The recent evolution of preservation solutions (Fukuse et al., 1996; Wittwer et al., 2005) and other techniques for organ procurement (Ardehali et al., 2003) facilitates more remote organ donation and the increased capability to use marginal donor lungs; however, preservation-related organ injury is still an unavoidable and sometimes critical problem (Meyers et al., 2005).

The preservation-related injury of the allograft, including ischemia-reperfusion injury imposed on lung allografts after transplantation, induces a substantial tissue inflammatory response resulting in a lung injury characterized by diffuse alveolar damage. Ischemia-reperfusion injury increases activation of vascular endothelial cells and releases a variety of chemoattractants, which help the activated neutrophils influx into the lung parenchyma with the coordination of adhesion molecules that are up-regulated during ischemia-reperfusion injury (Naka et al., 1997). During this nonspecific insult, it has been thought that neutrophils play a key role through a number of mechanisms including release of reactive oxygen species and proteolytic enzymes (Shiraishi et al., 1997).

Meanwhile, there have been several reports suggesting potent direct or indirect effects of activated neutrophils in the development of acute allograft rejection. Activated neutrophils release several factors such as CC chemokines that attract and activate antigen-presenting cells like dendritic cells (DCs), thus influence the priming of naïve antigen-specific T cells (Scapini et al., 2000; Chertov et al., 2000). It was recently shown that the neutrophil itself can take up antigens, transcribe and express the genes encoding major histocompatibility complex (MHC) molecules as well as co-stimulatory molecules, thereby behave as antigen presenting cells (Reali et al., 1996; Potter and Harding, 2001). Additionally, once neutrophils are exposed to inflammatory signals, activated neutrophils produce cytokines and chemokines to induce Th1 polarization of antigen specific T lymphocytes that influence the T-cell immune response (Graf et al., 2001). These observations suggest a possible linkage between neutrophils and the development of acute cellular rejection. See also Gilli et al. (2005), Grau et al. (1999, 2000), Schmouder et al. (1995) and Sibbring et al. (1998).

It would thus be useful to further understand the role of neutrophils in cellular rejection.

The present invention addresses that need.

### SUMMARY OF THE INVENTION

The inventors have discovered that allograft rejection can be inhibited or prevented by treating the allograft recipient with an inhibitor of neutrophil activation such as antileukinate (see Example).

The present invention is directed to methods of treating a mammal at risk for allograft rejection. The methods comprise treating the mammal with a compound that reduces an interaction between a CXC chemokine and a CXC receptor.

### BRIEF DESCRIPTION OF THE DRAWINGS

FIG. 1 is graphs showing radiographic aeration scores (score 0=opaque to score 6=normal-appearing lung). In Panel A, the group A treatment (antileukinate, early administration group) shows no significant differences at each time point between the control (Cont-A) and the treatment group. In panel B, the group B (antileukinate, continuous administration group) also shows no significant differences at each time point between the control (Cont-B) and the treatment group. The values are expressed as mean±standard deviation.

FIG. 2 shows representative chest radiographs and macroscopic views of the allografts. The upper panels show chest X-rays of allograft recipients on Day 7 after transplantation. Panel A shows a Group A recipient (antileukinate, early administration) (AS=6); Panel B shows a Group B recipient (antileukinate, continuous administration) (AS=5); Panel C shows a Control B allograft recipient (AS=1). The lower panels are photographs of the gross anatomy of (Panel D) a group A recipient; (Panel E) a group B recipient, and (Panel F) a Control B recipient. (AS=aeration score.)

FIG. 3 is representative photomicrographs of lung allografts at day 7 after transplantation (H & E, magnification ×200). Panel a shows a section from a Group A allograft recipient. Severe leukocyte infiltration and cuffing formation is evident in the perivascular space (RS=3). Panel b shows a section from a Group B allograft, and panel c shows a section from a Cont-B allograft showing weak rejection; perivascular infiltrates are present without extension into alveoli (RS=1). (RS=rejection score)

FIG. 4 is graphs showing the histologic rejection scores of lung allografts. Panel a shows a graph of Group A (antileukinate bolus injection group: white bar) and its control (untreated group: black bar). Panel b shows a graph of Group B (antileukinate continuous infusion group: shadow bar) and its control (untreated group: black bar). There is a significant difference in histologic rejection scores between group B and Cont-B (2.1±1.0 versus 3.3±0.5, p=0.018). The value was expressed as mean±standard deviation. The asterisk (*) mark indicates significant difference (p=0.018) in comparison with Cont-B.

FIG. 5 is graphs showing the number of neutrophils infiltrated into lung grafts. Panel a shows Group A (antileukinate bolus injection group: black squares) and its control (untreated group: white squares). Panel b shows Group B (antileukinate continuous infusion group: black triangles) and its control (untreated group: white triangles). The values are shown with the corresponding means. There is a significant difference in the number of neutrophils that have infiltrated the lung between group B and Cont-B.

### DETAILED DESCRIPTION OF THE INVENTION

The inventors have discovered that allograft rejection can be inhibited or prevented by treating the allograft recipient with an inhibitor of neutrophil activation such as antileukinate (see Example).

The present invention is directed to methods of treating a mammal at risk for allograft rejection. The methods comprise treating the mammal with a compound that reduces an interaction between a CXC chemokine and a CXC receptor.

As used herein, a chemokine is a small protein that guides the migration of cells toward increasing chemokine concentration. A CXC chemokine is a chemokine that has two conserved cysteines in the N-terminal domain that are separated by a single amino acid.

Preferably, the CXC chemokine is an ELR⁺ CXC chemokine, which is a CXC chemokine that has a glutamic acid-leucine-arginine (ELR) motif immediately before the first cysteine of the CXC motif. ELR⁺ CXC chemokines are known to specifically induce the migration of neutrophils. A preferred example of a ELR⁺ CXC chemokine is interleukin-8 (IL-8).

A CXC receptor is a transmembrane protein that interacts with CXC chemokines. For the instant methods, the preferred CXC receptor is a CXCR1 or CXCR2, which interact with ELR⁺ CXC chemokines.

In some aspects of these methods, the compound blocks the interaction between the CXC chemokine and the CXC receptor. Examples of such compounds are described, for example, in U.S. Pat. No. 5,965,536; Gordon et al., 2005; Kakutani et al., 1999; and White et al., 1998.

In other aspects of these methods, the compound reduces expression of the CXC chemokine. Non-limiting examples here are antisense, ribozymes and miRNA molecules that specifically inhibit transcription or translation of the CXC chemokine. Thus, the compound can be a nucleic acid having a region complementary to mRNA encoding the CXC chemokine.

In still other aspects, the compound reduces expression of the CXC receptor. Included here are antisense, ribozymes and miRNA molecules that specifically inhibit transcription or translation of the CXC receptor. As used herein, “miRNA” is any small, noncoding RNA (17-25 nt) that regulates gene expression by targeting mRNAs for translational repression, degradation, or both. Thus, the compound can be a nucleic acid having a region complementary to mRNA encoding the CXC receptor.

A preferred compound for these methods is an antileukinate comprising 6 or 7 amino acids comprising the sequence RRWWC. Antileukinate is defined herein as a peptide comprising 6 or 7 amino acids comprising the sequence RRWWC, able to inhibit the IL-8-CXCL8 interaction. See, e.g., U.S. Pat. No. 5,965,536. Preferably, the compound is an antileukinate comprising 6 or 7 amino acids comprising the sequence RRWWCR. More preferably, the antileukinate is RRWWCX, where X is any amino acid. Even more preferably, the antileukinate is RRWWCRX, where X is any amino acid. In the most preferred embodiments, the antileukinate is Ac-RRWWCR-NH₂.

Another preferred compound for these methods is SB225002. See, e.g., White et al., 1998. Still another preferred compound is JTE-607. See, e.g., Kakutani et al., 1999.

The compound of these methods can also be aptamer; alternatively, the compound can comprise an antibody binding site, e.g., a polyclonal or monoclonal antibody, an Fab fragment, or any recombinantly produced protein comprising an antibody binding site that binds to either the CXC chemokine or the CXC receptor, blocking the interaction.

These methods can be used with any allograft, including intraspecies and interspecies allografts. The allograft can be of any organ or tissue, including heart or liver. Preferably the allograft is a lung allograft or a kidney allograft.

The administration of the compound can follow any regimen appropriate for the particular application. The determination of dosing quantity and timing for any particular application can be determined by the skilled artisan without undue experimentation. In some embodiments, the mammal is treated with the compound before the allograft. In other embodiments, the mammal is treated with the compound after the allograft. Preferably, the mammal is treated with the compound both before and after the allograft. Additionally, the mammal may be undergoing allograft rejection when treated.

These methods are useful with any mammal. Preferably, the mammal is a human.

The invention methods preferably inhibits ischemia-reperfusion injury to the allograft, as antileukinate treatment is believed to do (Hirayama et al., 2006). The methods can also further comprise treatment with a second compound, wherein the second compound inhibits allograft rejection. Examples include anti-rejection drugs.

Preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the example.

### EXAMPLE

### Prevention of Neutrophil Migration Ameliorates Rat Lung Allograft Rejection

This Example is substantially published as Hirayama et al., 2006.

### Example Summary

Background. Chemokines activate and recruit specific leukocyte subpopulations. It was evaluated whether neutrophil migration, which can contribute to the development of ischemia-reperfusion injury, correlates with lung allograft rejection.

Methods. Orthotopic left lung allotransplantation was performed from Brown Norway (donor) to Fisher 344 (recipient) rats. The role of activated neutrophils in the development of allograft rejection is believed to be biphasic. Therefore, specific CXC receptor inhibition was used with antileukinate in two dosing regimens. Recipients were allocated into 4 groups: A (early administration: n=6) received two doses of antileukinate (a hexapeptide), 10.0 mg/kg intramuscularly 24 hours before and immediately after transplantation, B (continuous administration: n=8) animals continuously received antileukinate intraperitoneally (10.0 mg/kg/day) for 7 days after surgery. Experimental groups A or B were compared with their individual control that received saline alone (Cont-A and Cont-B). The progression of rejection was assessed radiographically. Each animal was sacrificed on day 7 for histologic evaluation of allograft rejection based on pathologic rejection grade.

Results. Histologic examination demonstrated significantly lower histologic rejection grade in group B compared with the untreated group (2.1±1.0 versus 3.3±0.5, p=0.018), whereas there was no significant difference in group A compared to the control group. There were no significant differences between the aeration scores of groups A or B as compared to their control groups.

Conclusions. The data suggest that neutrophils may play a promoting role in the development of allograft rejection, and blockage of neutrophil migration may suppress acute lung allograft rejection.

### Introduction

This Example describes experiments to determine whether suppression of neutrophil activation after allograft implantation might alleviate the molecular or cellular events in allograft rejection as well as ischemia-reperfusion injury.

Antileukinate is defined as a hexa-peptide, RRWWCR with acetylated amino-terminus and amidated carboxyl-terminus (Ac-RRWWCR-NH₂), which is a potent inhibitor of interleukin-8 (IL-8)/CXCL8 and growth related oncogene-α (GROα)/CXCL1 binding to receptors on activated neutrophils. Antileukinate is known to specifically inhibit IL-8-induced neutrophil chemotaxis and enzyme release (Hayashi et al., 1995) and has been considered a potent neutrophil activation inhibitor with the capability to protect lung tissue from acute lung injury induced by drugs (Hayashi et al., 2003) or sepsis (Lin et al., 2005). These findings suggest that antileukinate or its derivatives might prove useful in lung allograft reperfusion injury caused by activated neutrophils due to the unrestrained action of CXC-chemokines. This study is designed to investigate whether suppression of neutrophil activation during ischemia-reperfusion injury by this novel chemokine receptor antagonist affects the development and severity following acute lung allograft rejection.

### Materials and Methods

Animals. Inbred, male, specific-pathogen-free Brown Norway (BN, RT1ⁿ) and F344 (RT1^{1vl}) rats, weighing 250 to 300 g, were purchased from Kyudo Co., Ltd. (Tosu, Japan) and SLC Japan, Inc. (Shizuoka, Japan), respectively. Animals were cared for in compliance with The Principles of Laboratory Animal Care, formulated by the Institute of Laboratory Animal Resources, and the Guide for the Care and Use of Laboratory Animals, prepared by the National Academy of Sciences and published by the Institute for Laboratory Animal Research (National Academy Press, revised 1996).

Transplant Model. Rat left-lung transplantation was performed in a RT1 (MHC) incompatible donor-recipient combination from a BN donor into a F344 recipient. Complete graft rejection in this severely mismatched combination would be expected histologically and radiographically on day 7 postoperatively. The orthotopic left-lung transplantation procedure used in this study was a modification of the ‘cuff’ technique for vascular anastomosis (Mizuta et al., 1989). In brief, the lung grafts were flushed and immersed in ice-cold normal saline solution before implantation. Cuffs with internal diameters of 1.65 mm and 2.20 mm for pulmonary arterial and venous anastomosis, respectively, were used. The left main bronchus was anastomosed using 8-0 prolene running sutures. In rats, radiographic assessment of a left-lung transplantation graft can be obscured by the right lung. Therefore, a postcaval lobectomy on the recipient's right lung was performed immediately after implantation so as to facilitate radiographic assessment of the left-lung allograft. Rats with acute allograft dysfunction, as seen by an abnormal chest radiography immediately following implantation, were judged as technical failures and excluded from further analysis.

Agent. A hexa-peptide, Ac-RRWWCR-NH2 (antileukinate), was synthesized by the t-boc method and purified by SynPep Corporation (Dublin, Calif.).

Experimental groups. Recipient animals were divided into 4 groups (Table 1). Animals from group A (early administration; n=6) received 0.5 ml of phosphate-buffered saline (PBS) solution containing a 10.0 mg/kg concentration of antileukinate intramuscularly 24 hours before (day −1) and immediately after transplantation (day 0). The control group against group A (Cont-A) received 0.5 ml of PBS in a similar fashion. Allograft recipients from group B (continuous administration; n=8) were given continuous antileukinate administration intraperitoneally for 7 days after transplantation, starting immediately after surgery using a micro-osmotic pump which was implanted into the peritoneal cavity two hours before lung transplantation. Antileukinate was released into the recipient's peritoneal cavity continuously at a dose of 10.0 mg/kg/day from days 0 to 7. Animals from Cont-B served as the control group against group B receiving the same amount of PBS without antileukinate in the same manner.

TABLE 1
Experimental groups

| | | Antileukinate | | | | |
|---|---|---|---|---|---|---|
| Groups | n | treatment | Administration | Dose and timing | TST (min) | GIT (min) |
| A | 6 | Yes | 2 doses of | 10.0 mg/kg/day, | 140.3 ± 7.4 | 109.7 ± 3.6 |
| | | | bolus inj; im | 24 hrs before and | | |
| | | | | immediately | | |
| | | | | after Tx | | |
| Cont-A | 6 | No | * | | 130.8 ± 7.9 | 104.0 ± 7.6 |
| B | 8 | Yes | continuous, ip | 10.0 mg/kg/day | 140.6 ± 24.5 | 113.3 ± 24.0 |
| | | | | from Days 0 to 7 | | |
| Cont-B | 8 | No | * | | 138.8 ± 17.6 | 107.3 ± 15.2 |

* control groups were given same amount of PBS against each AL treated groups with same fashion, im: intramuscular, ip: inrraperitoneal using micro-osmotic pumps, Tx: transplantation, PBS: phosphate-buffered saline, TST: total surgical time (time from the beginning of recipients thoracotomy to the end of surgery), GIT: graft ischemic time (time from donor lung flush to its reperfusion when implantation completed).

Implantation of micro-osmotic pumps for groups B and Cont-B. Micro-osmotic pumps (model 2001; Alzet Corp., distributed by Charles River Laboratories, Sulzfeld, Germany) were used for intraperitoneal delivery of antileukinate. Pumps were preincubated for 4 hours at 37° C. in sterile saline according to the manufacturer's instructions and subsequently filled with 200 μl of antileukinate solution (104.2 μg/μl in phosphate-buffered saline, pH 7.4). After rats were anesthetized, an approximately 1 cm median laparotomy was performed and the pump was implanted in the abdominal cavity.

Radiographic evaluation. Transplanted lungs were serially assessed every other day by chest roentgenogram under spontaneous ventilation with halothane for sedation. Each chest roentgenogram was graded according to a previously published aeration score (AS) (Prop et al., 1987) by a blinded observer. Briefly, it was evaluated and graded 0 for an opaque lung, and up to 6 for a normal-appearing lung.

Histological evaluation. Allograft lungs were harvested on day 7 after transplantation. Acute rejection was evaluated histologically and assigned a rejection score (RS) based on the clinical international working formulation: IWF grade 0, no significant abnormality; grade 1, scattered infrequent perivascular mononuclear infiltration; grade 2, frequent perivascular mononuclear infiltration; grade 3, extension of mononuclear infiltration into alveolar septum/space; and grade 4, diffuse perivascular, interstitial, and air space infiltration of mononuclear cells. The number of neutrophils was counted in a blinded fashion in 10 random fields per slide at ×1000 magnification.

Statistical analysis. The data were expressed as mean±standard deviation. Analysis of intergroup differences by radiographic and histological scores was performed using the Mann-Whitney U test. Differences were considered significant if the p value was less than 0.05.

### Results

All animals survived the transplantation procedure. There were no significant differences in total surgical time (TGT: time from beginning of recipient's thoracotomy to the end of surgery) and graft ischemic time (GIT: time from donor lung perfusion to its reperfusion when implantation was completed) between each of the four groups (Table 1).

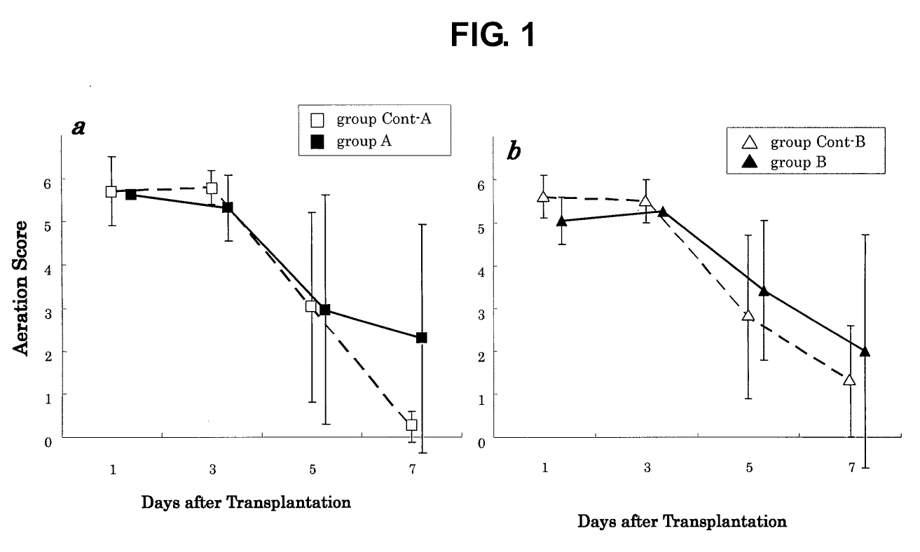

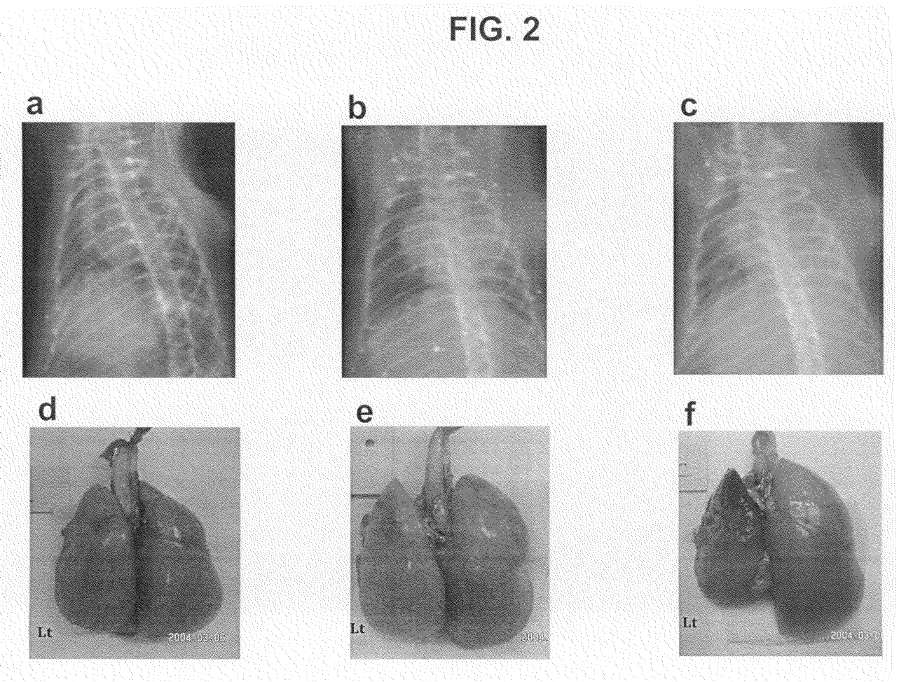

Radiographic findings. Allograft recipients from all four groups demonstrated excellent radiographic findings with no sign of rejection until day 3. On day 5 and thereafter, there was a progressive decrease in aeration score in all four groups with progression of rejection. No significant differences were observed between groups A and Cont-A, or B and Cont-B at any point in time (FIG. 1). On day 7 after transplantation, while the aeration scores for groups A and B were not statistically significantly different the aeration scores of the antileukinate treated groups tended to be better than the corresponding controls (AS on day 7, group B and Cont-B; 2.6±2.1 vs 1.6±1.3, p=0.16). Representative chest radiographs from groups A, B, and Cont-B are shown on FIG. 2 (upper panel). The AS for each of the 3 radiographs was evaluated as 6, 5, and 1, respectively.

Macroscopic findings of the allograft. The allograft lung from both Cont-A and Cont-B showed significant hepatinization, scattered hemorrhage, massive atelectasis, and complete consolidation without aeration compared with the native lung. In contrast, the allograft from groups A or B demonstrated macroscopically adequate ventilation, akin to the contralateral native right lung. Gross images of the representative allografts from groups A, B and Cont-B on day 7 after transplantation are shown in FIG. 2 (lower panel).

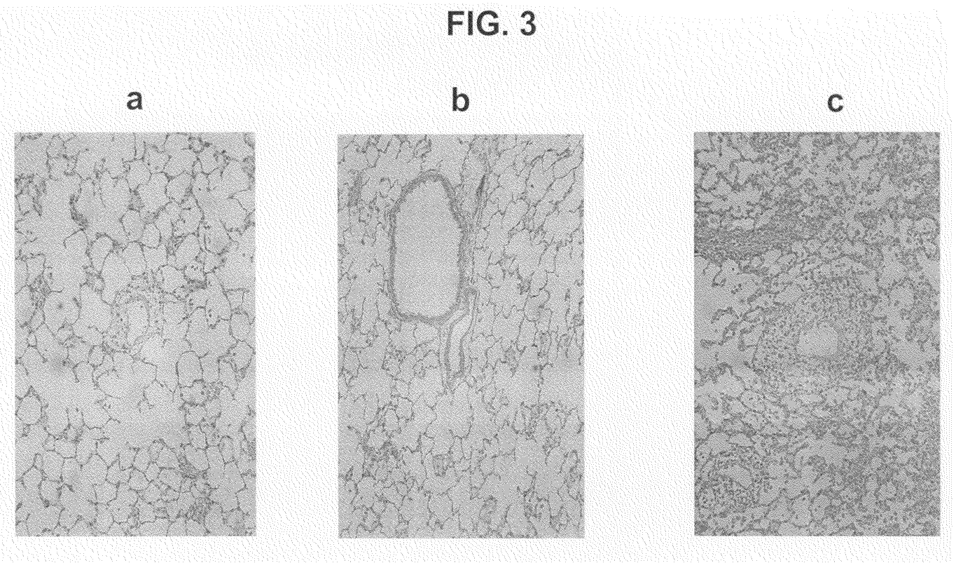

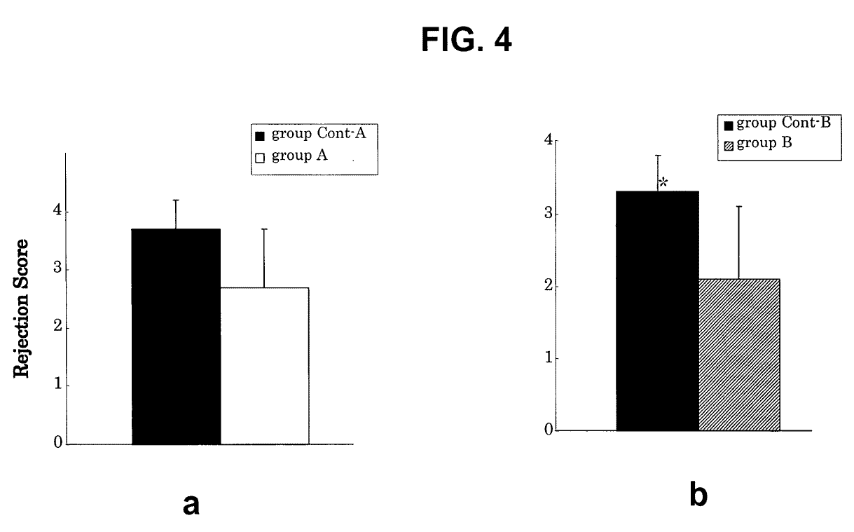

Microscopic finding and rejection score. Allografts from groups Cont-A and Cont-B showed diffuse perivascular, interstitial, and air space infiltration of mononuclear cells and were judged as the destructive phase of acute rejection (RS=4). In contrast, histology of allografts in groups A and B demonstrated only minimal perivascular infiltration of mononuclear cells around the pulmonary artery, indicating slight acute rejection (RS=1). Microscopic findings of the representative allografts from groups A, B, and Cont-B on day 7 at the time of sacrifice are presented in FIG. 3. The histologic rejection score, evaluated by means of IWF on day 7 demonstrated significantly less rejection in group B compared with its control (Cont-B) (2.1±1.0 versus 3.3±0.5, p=0.018), however, there was no significant difference between groups A and Cont-A (2.7±1.0 versus 3.7±0.5, p=0.078) (FIG. 4).

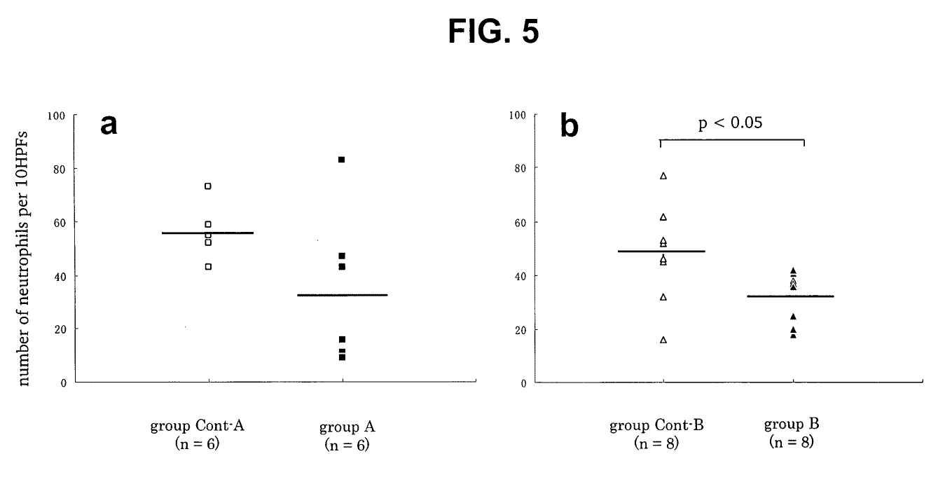

The effects of antileukinate on the allograft. To investigate whether or not antileukinate acts on the allograft, we blindly counted number of neutrophils in the graft. The result is shown in FIG. 5. The neutrophil count was significantly decreased to 32.1±9.6/10 HPFs in group B compared to 47.9±18.4/10HPFs in its control group (Cont-B) (p=0.046). However, although the trend was similar in the early administration groups, there was no significant difference between group A and Cont-A (34.8±28.7/10HPFs versus 55.7±10.0/10HPFs, p=0.093).

### Discussion

Acute allograft rejection is basically primed by alloantigen-specific T cells that respond to specific donor antigen, and infiltrate into the graft where they are activated to exhibit immune functions that mediate cellular immunologic reactions. However, the immunologic processes that initiate allograft rejection of a solid organ allograft are complex, involving the interplay of cells, antigens, and cytokines or chemokines.

Meanwhile, there have been many reports suggesting that preservation-related injury including ischemia-reperfusion injury causes not only early organ dysfunction after transplantation, but also increased frequency or severity of acute allograft rejection (Ettinger et al., 1990; Howard et al., 1990). Local production of cytokine in the allograft such as interleukin (IL)-1 and -2, tumor necrosis factor-alpha (TNF-α), and interferon-gamma (IFN-γ) are increased during ischemia-reperfusion injury and such mediators play an important role in initiating increased T lymphocyte and natural killer cell cytotoxicity (Yang and Welsh, 1986; Chang et al., 1990; Rau et al., 1991; Adoumie et al., 1992). Furthermore, these cytokines are known to be important regulators of MHC antigen expression and allograft immunogenicity. In addition, intracellular adhesion molecule expression has been shown to be up-regulated during reperfusion, with increased adhesion and migration of alloreactive lymphocytes that trigger the allograft rejection process (Horgan et al, 1991; Fuggle et al., 1993; Morita et al., 2001; Pelletier et al., 1993).

The neutrophil, which has been studied mainly in relation to ischemia-reperfusion injury, such as the source of reactive oxygen intermediates, plays a crucial role in ischemia-reperfusion injury development. Neutrophils are among the earliest leukocytes to traffic into inflammatory sites and are potent amplifiers of early inflammation in the allograft after organ transplantation. Reperfusion of ischemic tissues quickly induces production of neutrophil chemoattractants, such as GROα or macrophage inflammatory protein-2 (MIP-2) which enhance neutrophil infiltration into the parenchyma of transplanted tissues. In addition to chemokines, neutrophil trafficking into parenchymal tissues can be directed by many other mediators, such as C5a (Baldwin et al., 2001).

The important role of neutrophils and their potential effects in the development of the cellular immunologic reaction is well recognized. However, the role of neutrophils on macrophage recruitment and activation (Maus et al., 2002) and the direct and indirect effects of neutrophils on adaptive T-cell responses have only recently been recognized (Chertov et al., 2000). Activated neutrophils stimulate antigen presenting cells such as DCs by producing CC chemokine ligands CCL3 (MIP1α), CCL4 (MIP1β), CCL5 (RANTES) and CCL20 (MIP-3α) which chemoattract immature DC (Buonocore et al., 2004). Neutrophils can induce DC maturation as indicated by up-regulating key co-stimulatory molecules, including CD40, CD80 and CD86 (Bennouna et al., 2003). In addition, it is reported that neutrophils behave as antigen presenting cells by antigen uptake, transcribing and expressing the genes encording MHC class I and class II molecules, and inducing T-cell dependent immune responses (Ashtekar and Saha, 2003). Furthermore, neutrophils are endowed with cross-presentation ability as they can process exogenous antigens via an alternate MHC class I pathway and subsequently stimulate CD8+ T cells. Once exposed to inflammatory signals, activated neutrophils produce cytokines and chemokines that influence T-cell differentiation. In several models, neutrophils recruited after infection indeed produced IFN-γ, TNF-α and IL-12, and induced Th1 polarization of antigen specific T lymphocytes that influence T-cell immune responses (Graf et al., 2001; Bliss et al., 2000; Chen et al., 2001; Chen et al., 2000; Tateda et al., 2001). Those findings suggest that neutrophils influence both the “induction” phase when alloantigen is recognized by the recipient immune system and the “effector” phase when specific T-cell proliferation is accelerated (Buonocore et al., 2004). These observations and suggestions cause us to ascertain the linkage between neutrophils and the development of acute cellular rejection. Thus, these two experimental settings were prepared to investigate the effect of neutrophil suppression, either through the induction or effector phases, on the development of allograft rejection. In addition, antileukinate is a hexapeptide, and would be expected to be cleared rapidly from the bloodstream. Part of the reason for the disappearance from the plasma is binding to the CXC receptors on the neutrophils. Neutrophils in the circulation are short lived (8-20 hr) after which they undergo spontaneous apoptosis and are cleared (Hu et al., 2004). For these reasons, we have used two different administration regimen. In the A group, recipients were given an Antileukinate bolus to suppress neutrophil activation during the induction phase. In contrast, animals from group B were administered antileukinate continuously to investigate the effect of neutrophils in the effector phase.

All left lung allografts were well-aerated during the first 3 days after transplantation. Then, acute rejection was detected by means of decreased AS in all four groups. The data from chest radiographs on the basis of semiquantitative aeration score demonstrated no significant difference between group A and its control Cont-A, or B and Cont-B during the entire observation period. However, on day 5 and thereafter, the aeration score progressively decreased in the Cont-B whereas group B showed less of a decrease in the aeration score than Cont-B. Significant alleviation of acute allograft rejection on group B against its control was confirmed histologically by the rejection score based on the IWF scoring system. However, there was no significant difference in the degree of acute rejection in both radiographic and histologic findings against its control in group A, which received antileukinate only on the induction phase after transplantation. Interestingly, consistent with its CXC receptor inhibitory activity, continuous administration of antileukinate resulted in significantly less neutrophils within the graft. While the trend was similar in the early antileukinate administration groups, this did not reach the level of significance.

These findings suggest that antileukinate may effectively suppress the progression of rejection during the effector phase. The mechanism of action of Antileukinate on the suppression of acute lung allograft rejection was not fully confirmed in this experimental setting. However, it is thought that neutrophil suppression in the induction phase only is not sufficient to suppress the rejection process. Long term suppression of neutrophil activation might be necessary to suppress the “neutrophil-dependent allograft rejection process”.

Although antileukinate had only a partial beneficial effect in this experiment, inhibition of neutrophil activation may contribute in preventing allograft rejection. We could expect better results using antileukinate efficiently in combination with other immunosuppressive drugs. In conclusion, our data indicate that neutrophils promote the development of allograft rejection and that inhibition of neutrophil activation and migration suppresses acute rat lung allograft rejection.

### REFERENCES

Adoumie R, Serrick C, Giaid A, et al: Early cellular events in the lung allograft. Ann Thorac Surg 1992; 54:1071-7
Ardehali A, Laks H, Russell H, Levine M, Shpiner R, Lackey S, Ross D: Modified reperfusion and ischemia-reperfusion injury in human lung transplantation. J Thorac Cardiovasc Surg 2003; 126:1929-34
Ashtekar A R, Saha B: Poly's plea: membership to the club of APCs. Trends Immunol 2003; 24(9):485-90
Baldwin W M, Larsen C P, Fairchild R L: Innate immune responses to transplants: a significant variable with cadaver donors. Immunity 2001; 14:369-376
Bennouna S, Bliss S K, Curiel T J, Denkers E Y: Cross-talk in the innate immune system: neutrophils instruct recruitment and activation of dendritic cells during microbial infection. J Immunol 2003; 171(11):6052-8
Bliss S K, Butcher B A, Denkers E Y: Rapid recruitment of neutrophils containing prestored IL-12 during microbial infection. J Immunol 2000; 165(8):4515-21
Buonocore S, Surquin M, Moine A L, Abramowicz D, Flamand V, Goldman M: Amplification of T-cell responses by neutrophils: relevance to allograft immunity. Immunology Letters 2004; 94: 163-6
Chang S C, Hsu H K, Perng R P, Shiao G M, Lin C Y: Increased expression of MHC class II antigens in rejecting canine lung allograft. Transplantation 1990; 49: 1158-63
Chen L, Watanabe T, Watanabe H, Sendo F: neutrophil depletion exacervates experimental Chagas' disease in BALB/c, but protects C57BL/6 mice through modulating the Th1/Th2 dichotomy in different directions. Eur J Immunol 2001; 31(1):265-75
Chen L, Zhang Z, Sendo F: Neutrophils play a critical role in the pathogenesis of experimental cerebral malaria. Clin Exp Immunol 2000; 120(1): 125-33
Chertov O, Yang D, Howard O M, Oppenheim J J: Leukocyte granule proteins mobilize innate host defenses and adaptive immune responses. Immunol Rev 2000; 177:68-78
Ettinger S L, McLoughlin M G, Scudamore C H, Miller R R, Keown P A: Effects of recovery from ischemic injury on class I and class II MHC antigen. Transplantation
Fuggle S V, Sanderson J B, Gray D W R, Richardson A, Morris P J: Variation in expression of endothelial adhesion molecules in pretransplant and transplanted kidneys: correlation with intragraft events. Transplantation 1993; 55:117-123
Fukuse T, Hirata T, Ueda M, Hitomi S, Wada H: Effects of Euro-Collins, University of Wisconsin, and new extracellular-type trehalase-containing Kyoto solutions in an ex vivo rat lung preservation model. Transplantation 1996; 62:1212-1217
Gilli U O et al.: Human polymorphonuclear neutrophils are recruited by porcine chemokines acting on CXC chemokine receptor 2, and platelet-activating factor. Transplantation 2005; 79: 1324-31
Gordon J R et al.: The combined CXCR1/CXCR2 antagonist CXCL8₍₃₋₇₄₎K11R/G31P blocks neutrophil infiltration, pyrexia, and pulmonary vascular pathology in endotoxemic animals. J Leucocyte Biol 2005:1265-72
Graf M R, Prins R M, Merchant R E: IL-6 secretion by a rat T9 glioma clone induces a neutrophil-dependent antitumor response with resultant cellular antiglioma immunity. J Immunol 2001; 166(1):121-9
Grau V et al.: Time course of chemokine expression in acutely rejecting rat kidneys. Transplant Proc 1999; 31:1571.
Grau V et al.: Chemokine expression during acute rejection of rat kidneys. Scand J Immunol 2000; 51:435-40.
Hayashi S, Kurdowska A, Miller E J, Albright M E, Girten B E, Cohen A B: Synthetic hexa- and heptapeptides that inhibit IL-8 from binding to and activating human blood neutrophils. J Immunol 1995; 154:814-824
Hayashi S, Yatsunami J. Fukuno Y, Kawashima M, Miller E J: Antileukinate, a hexapeptide inhibitor of CXC-chemokine receptor, suppresses bleomycin-induced acute lung injury in mice. Lung 2003; 180:339-348
Hirayama S, Shiraishi T, Shirakusa T, Higuchi T, Miller E J: Prevention of neutrophil migration ameliorates rat lung allograft rejection. Mol Med 2006; 12:208-213
Horgan M J, Ge M, Gu J, Tothlein R, Malik A B: Role of ICAM-1 in neutrophil mediated lung vascular injury following occlusion and reperfusion. AM J Physiol 1991; 261:H1578-84
Howard T K, Klintmalm G B G, Cofer J B, Husberg B S, Goldstein R M, Gonwa T A: The influence of preservation injury on rejection in the hepatic transplantation recipient. Transplantation 1990; 49:103-7
Hu M, Miller, E J, Lin X, Simms H H: Transmigration across a lung epithelial monolayer delays apoptosis of polymorphonuclear leukocytes. Surgery 2004; 135 (1):87-98
Kaktani et al. L JTE-607, a novel inflammatory cytokine synthesis inhibitor without immunosuppression, protects from endotoxic shock in mice. Inflamm res 1999; 48:461-8
Lin X, Yang H, Sakuragi T, et al: α-Chemokine receptor blockade reduces high mobility group box 1 (HMGB1) protein induced lung inflammation and injury and improves survival in sepsis. Am J Physiol Lung Cell Mol Physiol 2005; 1289(4):L583-90
Maus U, von Grote K, Kuziel W A, et al: The role of CC chemokine receptor 2 in alveolar monocyte and neutrophil immigration in intact mice. Am J Respir Crit Care Med 2002; 166:268-273
Meyers B F, Morena M, Sweet S C, et al: Primary graft dysfunction and other selected complications of lung transplantation: A single-center experience of 983 patients. J Thorac Cardiovasc Surg 2005; 129:1421-29
Mizuta T, Kawaguchi A T, Nakahara K, Kawashima Y: Simplified rat lung transplantation using cuff technique. J Thorac Cardiovasc Surg 1989; 97:578-81
Morita K Miura M, Paolone D R, Engeman T M, Kapoor A, Remick D G, Fairchaild R L: Early chemokine cascades in murine cardiac grafts regulate T cell recruitment and progression of acute allograft rejection. J Immunol 2001; 167:2979-2984
Naka Y, Toda K, Kayano K, Oz M C, Pinsky D J: Failure to express the P-selectin gene or P-selectin blockade confers early pulmonary protection after lung ischemia or transplantation. Proc Natl Acad Sci USA 1997; 94:757-761
Pelletier R P, Morgan C J, Sedmak D D, Miyake K, Kincade P W, Ferguson R M, Orosz C G: Analysis of inflammatory endothelial changes, including VCAM-1 expression, in murine cardiac grafts. Transplantation 1993; 55:315-320
Potter N S, Harding C V: Neutrophils process exogenous bacteria via an alternate class I MHC processing pathway for presentation of peptides to T lymphocytes. J Immunol Prop J, Wagenaar-Hilbers J P, Petersen A H, Wildenuur C R: Diagnosis of rejection in rat lung allografts by bronchoalveolar lavage. Transplant Proc 1987; 19:3779-80
Rao P N, Liu T, Snyder J T, et al: Reperfusion injury following cold ischemia activates rat liver Kuppfer cells. Transplant Proc 1991; 23:666-9
Reali E, Guerrini R, Moretti S, et al: Polymorphonuclear neutrophils pulsed with synthetic peptides efficiently activated memory cytotoxic T lymphocytes. J Leukoc Biol 1996; 60(2):207-13
Scapini P, Lapinet-Vera J A, Gasperini S, Calzetti F, Bazzoni F, Cassatella M A: The neutrophil as a cellular source of chemokines. Immunol Rev 2000; 177:195-203
Schmouder R et al.: Epithelial-derived neutrophil-activating factor-78 production in human renal tubule epithelial cells and in renal allograft rejection. Transplantation 1995; 59:118-24.
Shiraishi T, Kuroiwa A, Shirakusa T, et al: Free radical-mediated tissue injury in acute lung allograft rejection and the effect of superoxide dismutase. Ann Thorac Surg 1997; 64:821-5
Sibbring J et al.: Localization of C-X-C and C-C chemokines to renal tubular epithelial cells in human kidney transplants is not confined to acute cellular rejection. Transpl Immunol 1998; 6:203-8.
Tateda K, Moore T A, Deng J C, et al: Early recruitment of neutrophils determines subsequent T1/T2 host responses in a murine model of Legionella pneumophila pneumonia. J Immunol 2001; 166(5):3355-61
White J et al.: Identification of a potent, selective non-peptide CXCR2 antagonist that inhibits interleukin-8-induced neutrophil migration. J Biol Chem 1998; 273:10095-8
Wittwer T, Franke U F W, Fehrenbach A, et al: Experimental lung transplantation: impact of preservation solution and route of delivery. J Heart Lung Transplant 2005; 24:1081-90
Yang H, Welsh R M: Induction of alloreactive cytotoxic T cells by acute virus infection of mice. J Immunol 1986; 136:1186-93
U.S. Pat. No. 5,965,536

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of treating a mammal at risk for allograft rejection, the method comprising treating the mammal with a compound that reduces an interaction between a CXC chemokine and a CXC receptor.

2. The method of claim 1, wherein the CXC receptor is a CXCR1 or CXCR2.

3. The method of claim 1, wherein the CXC chemokine is an ELR⁺ CXC chemokine.

4. The method of claim 1, wherein the CXC chemokine is IL-8.

5. The method of claim 1, wherein the compound blocks the interaction between the CXC chemokine and the CXC receptor.

6. The method of claim 1, wherein the compound reduces expression of the CXC chemokine.

7. The method of claim 1, wherein the compound reduces expression of the CXC receptor.

8. The method of claim 1, wherein the compound is an antileukinate comprising 6 or 7 amino acids comprising the sequence RRWWC.

9. The method of claim 1, wherein the compound is an antileukinate comprising 6 or 7 amino acids comprising the sequence RRWWCR.

10. The method of claim 8, wherein the antileukinate is RRWWCX, where X is any amino acid.

11. The method of claim 8, wherein the antileukinate is RRWWCRX, where X is any amino acid.

12. The method of claim 8, wherein the antileukinate is Ac-RRWWCR-NH₂.

13. The method of claim 1, wherein the compound is SB225002.

14. The method of claim 1, wherein the compound is an aptamer or comprises an antibody binding site.

15. The method of claim 1, wherein the compound is JTE-607.

16. The method of claim 1, wherein the compound is a nucleic acid having a region complementary to mRNA encoding the chemokine or the receptor.

17. The method of claim 16, wherein the nucleic acid is an miRNA, an antisense, or a ribozyme specific for the chemokine or the receptor mRNA.

18. The method of claim 1, wherein the allograft is interspecies.

19. The method of claim 1, wherein the allograft is a heart or liver.

20. The method of claim 1, wherein the allograft is a lung.

21. The method of claim 1, wherein the allograft is a kidney.

22. The method of claim 1, wherein the mammal is treated with the compound before the allograft.

23. The method of claim 1, wherein the mammal is treated with the compound after the allograft.

24. The method of claim 1, wherein the mammal is treated with the compound both before and after the allograft.

25. The method of claim 1, wherein the mammal is undergoing allograft rejection when treated.

26. The method of claim 1, wherein the mammal is a human.

27. The method of claim 1, wherein the treatment inhibits ischemia-reperfusion injury to the allograft.

28. The method of claim 1, wherein the patient is treated with a second compound, wherein the second compound inhibits allograft rejection.
